(19) United States
(12) Patent Application Publication
(10) Pub. No.: US 2007/0093826 A1
(43) Pub. Date: Apr. 26, 2007

(54) Modular pedicle screw systems and methods of intra-operatively assembling the same

(76) Inventors: David T. Hawkes, Pleasant Grove, UT (US); David R. Warnick, Spanish Fork, UT (US); Michael Ensign, Salt Lake City, UT (US); Thomas Sweeney, Sarasota, FL (US)

Correspondence Address: RADER, FISHMAN & GRAUER PLLC, 10653 SOUTH RIVER FRONT PARKWAY, SUITE 150, SOUTH JORDAN, UT, 84095, US

(21) Appl. No.: 11/543,347

(22) Filed: Oct. 4, 2006

Related U.S. Application Data
(60) Provisional application No. 60/723,324, filed on Oct. 4, 2005.

(51) Int. Cl. A61F 2/30

(52) U.S. Cl. 606/61

(57) ABSTRACT

The systems, assemblies, devices and methods described herein provide a variety of ways to intra-operatively select and/or configure a pedicle screw system. The pedicle screw system components described herein may be readily interchangeable and may be made available to the surgeon as a kit, thus giving the surgeon greater options on structurally fusing, correcting, or otherwise operating on a patient's vertebrae. In one embodiment, a modular pedicle screw kit includes a pedicle screw having a threaded shaft coupled to a head portion and a plurality of tulip assemblies that may be intra-operatively assembled with the pedicle screw. Each tulip assembly includes a tulip body, a rod receiving portion, and a pedicle screw head coupling member that releasably couples the tulip body to the pedicle screw.

### RELATED APPLICATIONS

This application claims the benefit under 35 U.S.C. § 119(e) of U.S. Provisional Patent Application No. 60/723,324 filed Oct. 4, 2005 titled “Modular Pedicle Screw Systems and Methods of Interoperably Assembling the Same.” The provisional application is incorporated herein by reference in its entirety.

### FIELD

The present system and method relate generally to modular pedicle screw systems and methods of intra-operatively assembling the same.

### BACKGROUND

Pedicle screw systems are surgically implanted in a number of vertebrae during a spinal operation as an adjunct to fusion. Pedicle screw systems typically include the pedicle screw, a clamp device attached to the pedicle screw, and a rod or other structural member that is received and retained in the clamp device.

Before a spinal operation is scheduled, the surgeon or other trained professional typically performs a non-invasive review of at least a segment of the patient's spinal column. The non-invasive review is typically performed by acquiring an x-ray image, fluoroscopic image, magnetic resonance image (MRI), or other equivalent image of the segment of the patient's spinal column and then the surgeon, a radiologist, or perhaps both review the images. Based on the captured images and likely other factors, the surgeon will determine what type and size of pedicle screw systems will be used during the spinal operation on the patient. Once the type and size of the pedicle screw systems to be used is determined, the surgeon or other staff member will place an order with a manufacturer for the pedicle screw systems.

The manufacturer, once they have received the order, assembles each pedicle screw with the associated clamp device per the specifications identified in the order. It is common practice for the manufacturer to also assemble several “extra” pedicle screw systems comprising sizes that are slightly less and slightly greater than those actually ordered. The requested pedicle screw systems and the extra systems are often manually delivered to the surgery location and made readily available to the surgeon during the spinal operation.

One drawback of the traditional process is that either the surgeon has limited options on what type and even what size of pedicle screw systems to use once the surgery is in progress, i.e., intra-operative; or, the manufacturer is required to provide additional inventory at an exorbitant cost to the manufacturer. Specifically, the traditional state of the art provides that the clamp device and the pedicle screw are pre-assembled, which prevents the surgeon from exchanging or otherwise manipulating the components intra-operatively. For example, after the surgeon gets an intra-operative look at the patient's spinal column after opening the surgical site, the surgeon may decide that a substantially different type or size pedicle screw system is required. This situation may be more acute when the patient is undergoing a follow-up surgery to repair existing hardware, for example.

The pre-operative images typically obtained of the patient only provide a limited amount of information. Only when the surgeon is actively viewing the patient's spinal column can the surgeon have all the necessary information to determine what type and size of pedicle screw systems would be most appropriate for the patient.

Further, a preassembled screw and clamp device is more obtrusive in the surgical wound than just an implanted screw. So, a preassembled screw and clamp device complicate the surgical technique or inhibit certain surgical maneuvers.

### SUMMARY

According to one exemplary embodiment, the systems, assemblies, devices, and methods described herein provide a variety of ways to intra-operatively select and/or configure a pedicle screw system. The pedicle screw system components described herein may be readily interchangeable and may be made available to the surgeon as a kit, thereby providing the surgeon greater options on structurally fusing, correcting, or otherwise operating on a patient's vertebrae.

Particularly, according to one exemplary embodiment, a modular pedicle screw kit includes a pedicle screw having a threaded, elongated shaft coupled to a head portion; and a plurality of tulip assemblies configured to be installed on the head portion of the pedicle screw, each tulip assembly including a tulip body, a rod receiving portion, and a coupling member configured to releasably couple the tulip body to the pedicle screw after the pedicle screw is inserted in a vertebra.

In yet another exemplary embodiment, a method for intra-operatively assembling a pedicle screw kit includes inserting a pedicle screw into a vertebra, the pedicle screw having a threaded, elongated shaft coupled to a head portion; determining one type of tulip assembly to be installed on the head portion of the pedicle screw after the pedicle screw is inserted in the vertebra, each type of tulip assembly having a tulip body, a rod receiving portion, and a coupling member configured to compressively couple the tulip body to the pedicle screw; and coupling the one type of tulip assembly to the head portion of the pedicle screw.

The foregoing is a summary and thus contains, by necessity, simplifications, generalizations and omissions of detail. Consequently, those skilled in the art will appreciate that the summary is illustrative only and is not intended to be in any way limiting. Other aspects, inventive features, and advantages of the devices and/or processes described herein, as defined solely by the claims, will become apparent in the non-limiting detailed description set forth herein.

### BRIEF DESCRIPTION OF THE DRAWINGS

The accompanying drawings illustrate various exemplary embodiments of the present system and method and are a part of the specification. Together with the following description, the drawings demonstrate and explain the principles of the present system and method. The illustrated embodiments are examples of the present system and method and do not limit the scope thereof.

FIG. 1A and 1B are a top plan view and a side elevational view, respectively, of an upper portion of a first type of pedicle screw, according to one exemplary embodiment.

FIG. 2A is a side elevational view of a second pedicle screw including a split ball, according to one exemplary embodiment.

FIG. 2B is a top, right, isometric view of the second pedicle screw with a split ball, according to one exemplary embodiment.

FIG. 2C is a comparison of side elevational views showing the split ball of FIG. 2B in two different positions on the second exemplary pedicle screw, according to one exemplary embodiment.

FIG. 3 is a side elevational view of a third type of pedicle screw, according to one exemplary embodiment.

FIG. 4A is a top, right isometric view of a first type of pedicle screw system, according to one exemplary illustrated embodiment.

FIG. 4B is an exploded, isometric view of a tulip assembly of the first type as illustrated in FIG. 4A, according to one exemplary embodiment.

FIG. 5A is a top, right isometric view of a second type of pedicle screw system, according to one exemplary embodiment.

FIG. 5B is a side elevational view of the second type of pedicle screw system of FIG. 5A, according to one exemplary embodiment.

FIG. 5C is a top, right, exploded, isometric view of the second type of pedicle screw system of FIG. 5A, according to one exemplary embodiment.

FIGS. 6A and 6B illustrate a side elevational view and a top, right, exploded, isometric view, respectively, of a third type of pedicle screw system, according to one exemplary embodiment.

FIGS. 7A and 7B illustrate a side elevational view and an exploded view, respectively, of a fourth type of pedicle screw system, according to one exemplary embodiment.

FIG. 8 is an exploded view of a fifth type of pedicle screw system, according to one exemplary embodiment.

FIG. 9 is a side view of a modular pedicle screw system or kit, according to one exemplary embodiment.

FIG. 10 is a flow chart illustrating a method for placing the modular pedicle screw system or kit of FIG. 9, according to one exemplary embodiment.

In the drawings, identical reference numbers identify similar elements or acts. The sizes and relative positions of elements in the drawings are not necessarily drawn to scale. For example, the shapes of various elements and angles are not drawn to scale, and some of these elements are arbitrarily enlarged and positioned to improve drawing legibility. Further, the particular shapes of the elements as drawn, are not intended to convey any information regarding the actual shape of the particular elements, and have been solely selected for ease of recognition in the drawings. Throughout the drawings, identical reference numbers designate similar but not necessarily identical elements.

### DETAILED DESCRIPTION

The present specification describes a system and a method for providing a modular pedicle screw system. Particularly, according to one exemplary embodiment, the present specification discloses a number of pedicle screws and/or tulip assemblies configured to be combined to create pedicle screw kits that may be advantageously modified and assembled intra-operatively. Further details of the present exemplary system and method will be provided below.

Unless the context requires otherwise, throughout the specification and claims which follow, the word “comprise” and variations thereof, such as, “comprises” and “comprising” are to be construed in an open, inclusive sense that is as “including, but not limited to.”

Reference throughout this specification to “one embodiment” or “an embodiment” means that a particular feature, structure or characteristic described in connection with the embodiment is included in at least one embodiment of the present assemblies, devices and systems. Thus, the appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The headings provided herein are for convenience only and do not interpret or modify the scope or meaning of the claimed invention.

The pedicle screw systems described herein include different types of pedicle screws and/or tulip assemblies that can be combined to create pedicle screw kits that may be advantageously modified and assembled intra-operatively. In addition, according to one exemplary embodiment, the resultant kits are configured to advantageously provide the surgeon with tulip assemblies that can be intra-operatively assembled to a pedicle screw during surgery. For example, according to one exemplary embodiment, a first type of tulip assembly may be desired on a first vertebra while a second type of tulip assembly is desired on an adjacent vertebra. The two different types of tulip assemblies may be combined to achieve a certain type of alignment, to provide a certain degree of strength, or for a variety of other reasons.

Another possible advantage of the present exemplary pedicle screw kits is that a surgeon, once he or she has the surgical site open, may determine that the original type of tulip assemblies selected based on any number of pre-operative images are not the desired type of tulip assemblies. Consequently another type of tulip assembly may be desired. With the present exemplary pedicle screw kit, the surgeon can make this decision even after the pedicle screws have been inserted into a patient's vertebrae and will still have the option of installing a number of different types of tulip assemblies.

In the following description, certain specific details are set forth in order to provide a thorough understanding of various embodiments of the present tools, assemblies, systems, and methods. However, one skilled in the relevant art will recognize that the tools, assemblies, systems, and methods may be practiced without one or more of these specific details, or with other methods, components, materials, etc. In other instances, well-known structures associated with surgical tooling, surgical implant devices, and surgical spinal techniques have not been shown or described in detail to avoid unnecessarily obscuring descriptions of the embodiments of the present assemblies, devices and systems.

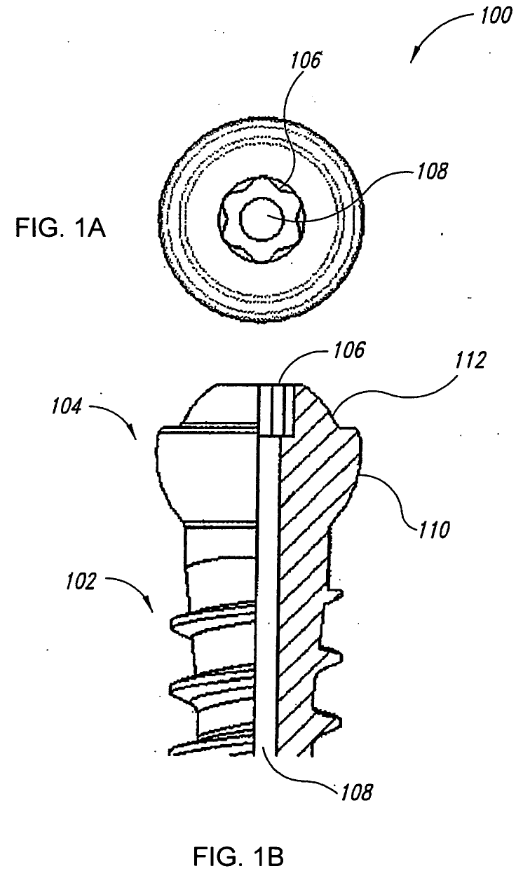

According to one exemplary embodiment, the present system and method includes a modular pedicle screw kit having a pedicle screw with a threaded elongated shaft coupled to a head portion, and a plurality of tulip assemblies configured to couple the head portion of the pedicle screw, each tulip assembly including a tulip body, a rod receiving portion, and a coupling member configured to releasably couple the tulip body to the pedicle screw after the pedicle screw is inserted in a vertebra. According to this exemplary embodiment, a surgeon may select from the various tulip assemblies intra-operatively without inserting different screws. Various details of the screw and tulip assemblies will be provided in detail below with reference to FIGS. 1A through 8.

Pedicle Screws

According to one exemplary embodiment of the present system and method, pedicle screws are surgically implanted into a patient's vertebrae during a spinal operation. The pedicle screws of the present system and method can take a variety of forms, but each form generally includes a threaded, elongated shaft coupled to a head portion. According to one exemplary embodiment, the threaded, elongated shaft may include, but is in no way limited to, a self-tapping thread. According to the present exemplary system and method, the head portion is sized and shaped to receive various types of tulip assemblies, which are described in detail below.

FIGS. 1A and 1B illustrate a pedicle screw (100) having a threaded, elongated shaft (102) coupled to a head portion (104), according to one exemplary embodiment. Further, as illustrated in FIGS. 1A and 1B, the pedicle screw (100) further includes a driving feature (106) and a cannulated opening (108). According to the present exemplary embodiment, the driving feature (106) allows the screw (100) to be engaged by an insertion tool including a driving member corresponding to the driving feature (106) for the initial placement of the screw into the vertebra as well as for adjustments made after installing one of the various tulip assemblies on the head portion (104) of the pedicle screw (100). Additionally, the illustrated cannulated opening (108) allows the pedicle screw (100) to be assembled over a k-wire (i.e., Kirschner wire). The k-wire (not shown) may have been pre-operatively positioned using a non-invasive imaging procedure such as, but in no way limited to, fluoroscopy.

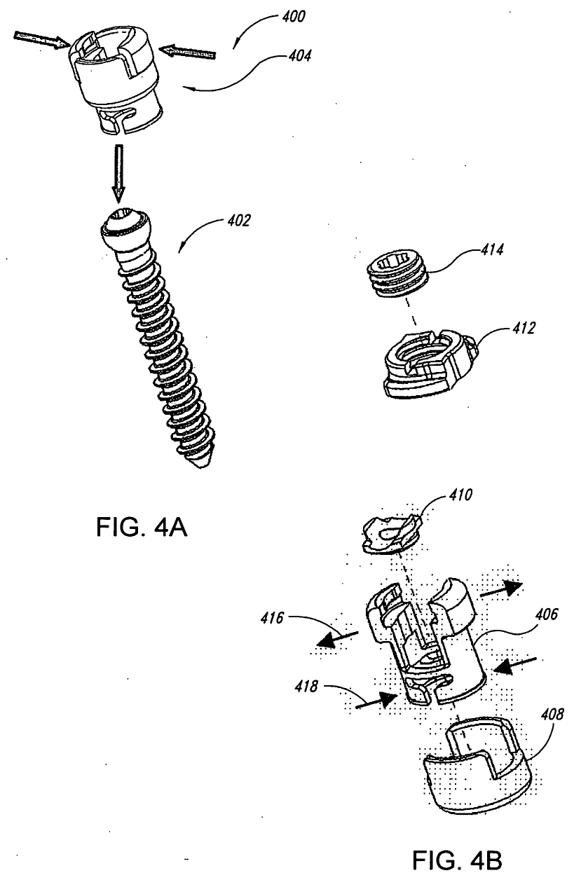

As illustrated in FIG. 1B, the head portion (104) of the pedicle screw (100) is a dual diameter head, according to one exemplary embodiment. For one type of tulip assembly, the first, larger diameter (110) of the head portion (104) mates with a spherical bore of a tulip body and the smaller diameter (112) mates with a rod saddle. This type of tulip assembly is described in detail below with reference to FIGS. 4A and 4B. One exemplary advantage of the dual diameter head portion (104) illustrated in FIG. 1B is that the dual diameter allows a rod saddle to sit lower on the pedicle screw (100), thus reducing the overall height of the tulip assembly, while maintaining concentric surfaces, which permits unconstrained movement of the head portion (104).

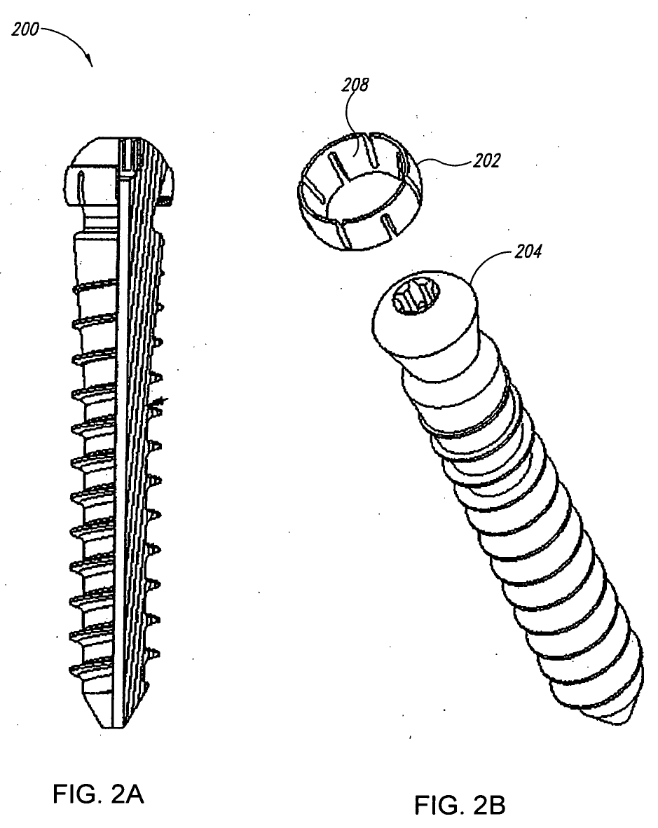

While FIGS. 1A and 1B illustrate one exemplary pedicle screw configuration, any number of pedicle screws may be used to perform the present exemplary system and method. For example, FIGS. 2A and 2B illustrate another type of pedicle screw (200) that is able to receive a particular type of tulip assemblies discussed below. According to one exemplary embodiment, the pedicle screw (200) may include a split ball (202) coupled to a head portion (204) of the screw. The split ball (202) can be coupled to the head portion (204) either pre-operatively or intra-operatively. According to one exemplary embodiment described in further detail below, the tulip body of one exemplary tulip assembly is configured to snap or compressively couple the split ball during surgery.

According to one exemplary embodiment, the split-ball (202) is free to translate along a tapered surface (206) of the head portion (204). The tapered surface (206) of the head portion interfaces with a reciprocal, inner tapered surface (208) of the split-ball (202). The mating or interfacing surfaces (206, 208) have an identical or substantially similar center of curvature, which may advantageously produce a less bulky assembly by decreasing the total height of the combination of the pedicle screw (200) and tulip assembly.

FIG. 2C illustrates that the split-ball (202) is expandable/contractible over the head portion (204) of the pedicle screw (200). Accordingly, the split ball (202) is shown in a low position (208) and a high position (210) on the head portion (202) of the pedicle screw (200). According to one exemplary embodiment, the split-ball (202) is in a contracted position when in the low position (208) and in an expanded position while in the high position (210).

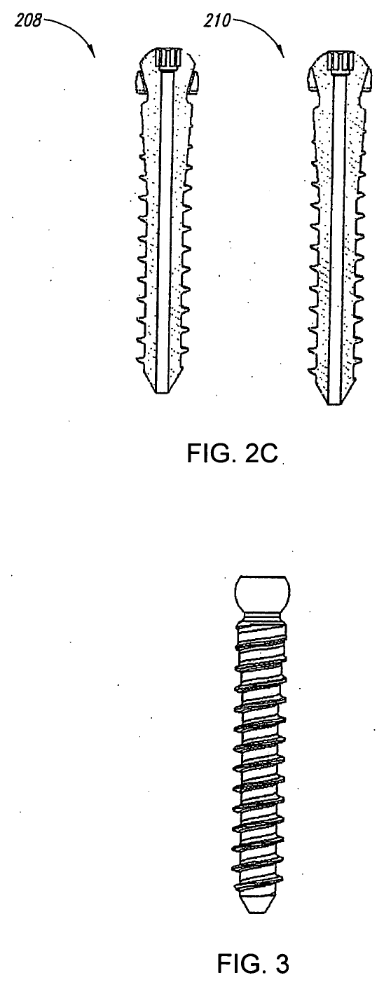

Further, FIG. 3 illustrates yet another type of pedicle screw (300), according to one exemplary embodiment. As illustrated in FIG. 3, the exemplary pedicle screw includes a spherical head portion (302) and an elongated, threaded shaft (304). According to one exemplary embodiment, the exemplary pedicle screw (300) shown in FIG. 3 can receive and be compressively coupled to a number of the types of tulip assemblies described below.

Pedicle Screw Systems

FIGS. 4A and 4B illustrate a modular pedicle screw system (400) including a pedicle screw (402) and a tulip assembly (404), according to one exemplary embodiment. According to the present exemplary system, it is understood that the pedicle screw (402) can be one of the pedicle screws described above with reference to FIGS. 1A through 3.

Referring now to FIG. 4B, the exemplary tulip assembly (404) comprises a tulip body (406), a hoop member (408), a rod saddle (410), a cap (412), and a set screw (414) as illustrated. The tulip assembly (404) illustrated in FIG. 4B is assembled by expanding a top portion of the tulip body (406) and compressing a bottom portion as indicated by the arrows (416, 418), respectively. The elasticity of the tulip body (406) holds the rod saddle (410) and hoop (408) in place. When placing the tulip body (406) over the head portion of the pedicle screw (402), the top portion of the tulip body can be compressed to open the bottom portion of the tulip body (406) and thereby receive the head portion of the pedicle screw (402) into a spherical bore of the tulip body (406).

In one exemplary embodiment, the hoop (408) is pre-operatively assembled with the tulip body (406) and serves to constrain the expansion of the tulip body (406). When the hoop (408) is slid over the flexible top portion of the tulip body (406), the bottom portion of the tulip body (406) is able to expand to receive the head portion of the pedicle screw (402), according to one exemplary embodiment. When the hoop (408) is slid down the tulip body (406), the hoop constrains the bottom portion of the tulip body (406) and thereby captures and retains the head portion of the pedicle screw (402). Once in a desired location, the hoop (408) is rotated to secure the hoop in the down position. At this point, the head portion of the pedicle screw (402) is at least translationally retained in the tulip body (406), but may be free to rotate therein.

Further, according to one exemplary embodiment, the rod saddle (410) is pre-operatively assembled with the tulip body (406) and serves to increase the surface area contact between the rod (not shown) and the pedicle screw (402). During assembly, the rod is inserted into the rod saddle (410) and the cap (412) is then coupled to the tulip body (406) to secure the rod.

When the cap (412) is rotated a quarter turn, a cam feature disposed on the cap expands or spreads the top portion of the tulip body (406) creating a clothes-pin effect, which causes the tulip body (406) to compressively couple the head portion of the pedicle screw (402). This clamping action provisionally fixes the tulip body (406) to the pedicle screw (402) before securing the rod in the rod saddle (410) with the cap (412) and the advancement of the set screw (414). The cap (412) can also include any number of features that interlock with the top portion of the tulip body (406) to resist post-operative splaying of the top portion of the tulip body (406). Additional aspects of the exemplary type of tulip assembly illustrated in FIG. 4A and 4B can be found in U.S. patent application Ser. No. 11/258,393, which reference is incorporated herein by reference in its entirety.

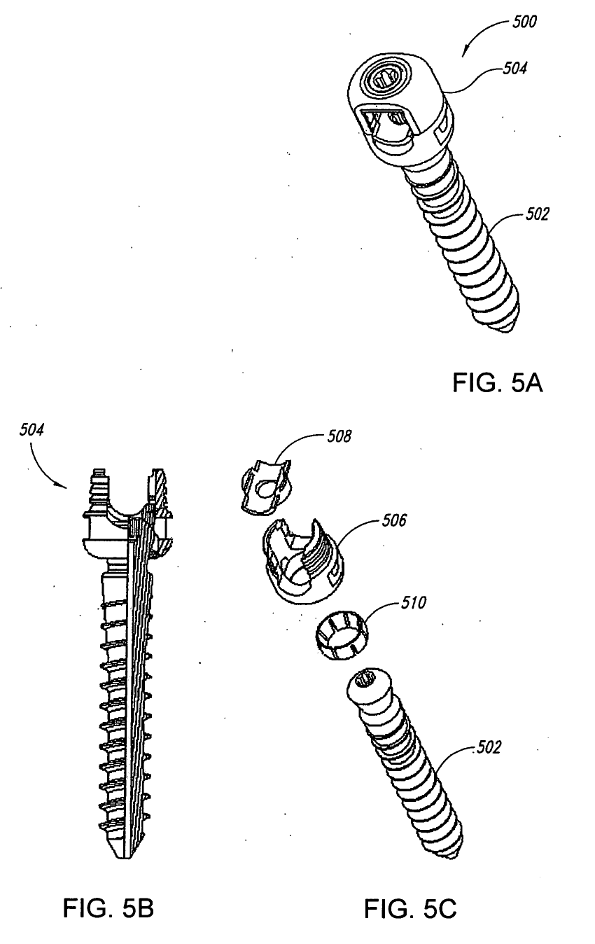

In addition to the exemplary screw system illustrated in FIGS. 4A and 4B, FIGS. 5A through 5C demonstrate another exemplary type of pedicle screw system (500) having a pedicle screw (502) and a tulip assembly (504) that may be interchangeably used by the present system and method, according to one exemplary embodiment. The pedicle screw system (500) illustrated in FIGS. 5A through 5C is designed for fixation of the spine in posterior lumbar fusion via minimally traumatic surgery (MTS) techniques. Similar to the other embodiments, the pedicle screw system (500) includes the pedicle screw (502) being configured to be inserted into the vertebrae, the tulip assembly (504) installed therewith, and then stabilizing rods (not shown) captured and retained by adjacent tulip assemblies (504). One purpose of the pedicle screw system (500) is to provide support to the spine while fusion of spinal segments occurs. It is understood that the pedicle screw (502) can be any one of the pedicle screws described in detail above, for example pedicle screw (200).

Referring to FIGS. 5B and 5C, the exemplary tulip assembly (504) includes a tulip body (506), a rod receiving member (508), and a split ball (510), according to one exemplary embodiment. The present exemplary tulip assembly (504) can be provided, during surgery, to a surgeon or other operating room staff member, as a two-piece assembly, which includes the tulip body (506) and the rod receiving member (508). The split ball (510) is pre-operatively positioned on the head portion of the pedicle screw (502).

Continuing with FIGS. 5B and 5C, the tulip body (506) includes a curved inner bore to receive the split ball (510). The rod receiving member (508) possesses sufficient travel within the tulip body (506) to allow the pedicle screw (502) to enter the tulip body (506) while the split ball (510) translates down a tapered surface of the head portion of the pedicle screw (502). This movement of the split ball (510) down the head portion of the pedicle screw (502) permits the split ball (510) to be manipulated over the head portion of the pedicle screw and to then compressively engage and retain the tulip body (506) on the pedicle screw (502). The pedicle screw system (500) is poly-axial in that the tulip body (506) can rotate about the head portion of the pedicle screw (503) to facilitate insertion of the rods in the rod receiving member (508). Additional aspects of the type of tulip assembly described herein can be found in U.S. patent application Ser. No. 11/259,748, which reference is incorporated by reference in its entirety.

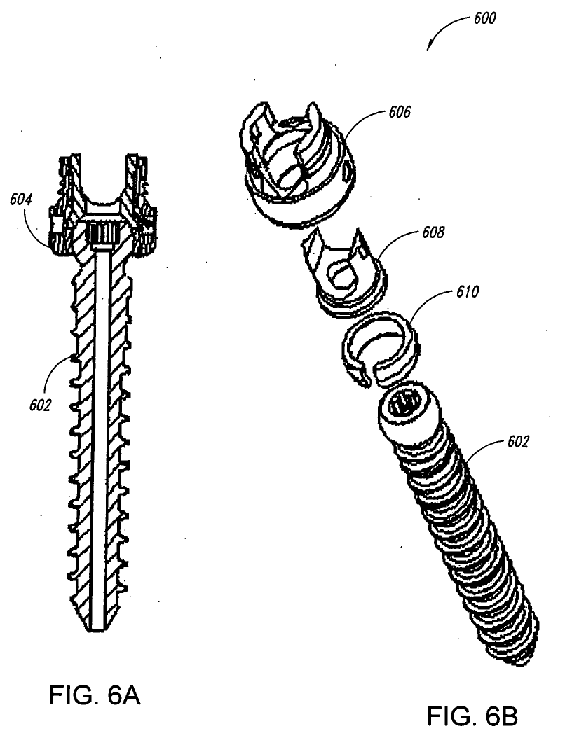

FIGS. 6A and 6B illustrate yet another pedicle screw system (600) including a pedicle screw (602) and a tulip assembly (604) that may be used to intra-operatively select a desired assembly, according to one exemplary embodiment. In this embodiment, the tulip assembly (604) is provided, during surgery, to a surgeon or other operating room staff member, as a three piece assembly, which includes a tulip body (606), a rod receiving member (608), and a split ring (610). The tulip body (606) includes an inner bore sized to receive and engage with the split ring (610), according to one exemplary embodiment. According to one exemplary embodiment, the inner bore is shaped to allow the head of the pedicle screw (602) to be received by the tulip body (606) and to permit the split ring (610) to expand, engage, and become compressively captured in the tulip body (606). According to this exemplary embodiment, the tulip body (606) further receives the rod receiving member (608), also referred to as a tulip inner. The pedicle screw system (600) illustrated in FIGS. 6A and 6B are poly-axial in that the tulip body (606) can rotate about the head portion of the pedicle screw (602) to facilitate rod installation and placement. Additional aspects of this type of tulip assembly as described herein can be found in U.S. patent application Ser. No. 11/259,748, which application is incorporated herein by reference in its entirety.

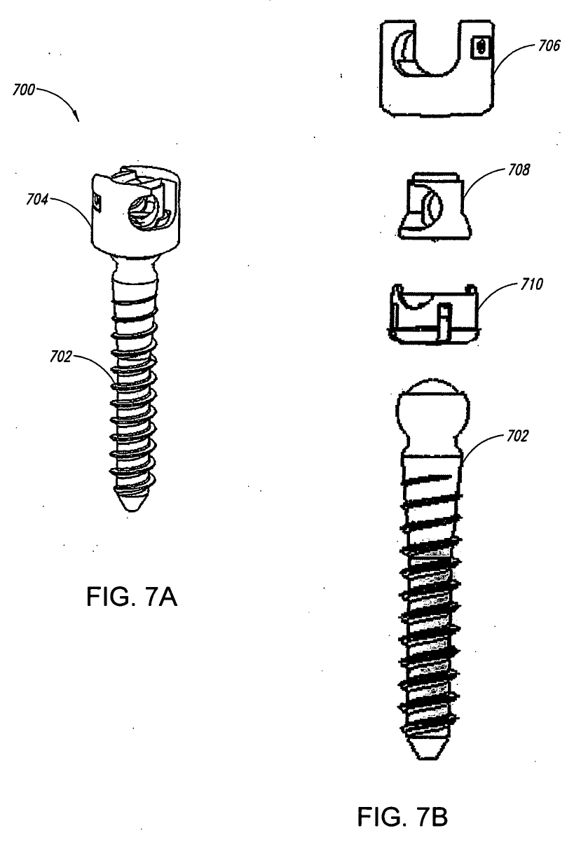

FIGS. 7A and 7B illustrate still another exemplary pedicle screw system (700) having a pedicle screw (700) and a tulip assembly (704) that may be used to intra-operatively select a desired assembly, according to one exemplary embodiment. In this embodiment, the tulip assembly (704) includes a tulip body (706), a rod receiving member (708), and a socket member (710). This illustrated embodiment performs the same function as the exemplary embodiment illustrated in FIGS. 6A and 6B; however, the present exemplary embodiment utilizes a different method for provisionally locking the angle of the tulip body (706) with respect to the pedicle screw (702). Particularly, according to the illustrated exemplary embodiment, provisional locking is achieved by rotating the rod receiving member (708) partially through its allowable rotation. Inclined planes on the rod receiving member (708) engage with tabs on the socket member (710) to push the socket member (710) onto the head portion of the pedicle screw (702), which provisionally locks the tulip body (706) onto the pedicle screw (702). Additional aspects of this exemplary tulip assembly as described herein can be found in U.S. patent application Ser. No. 11/258,831, which application is incorporated herein by reference in its entirety.

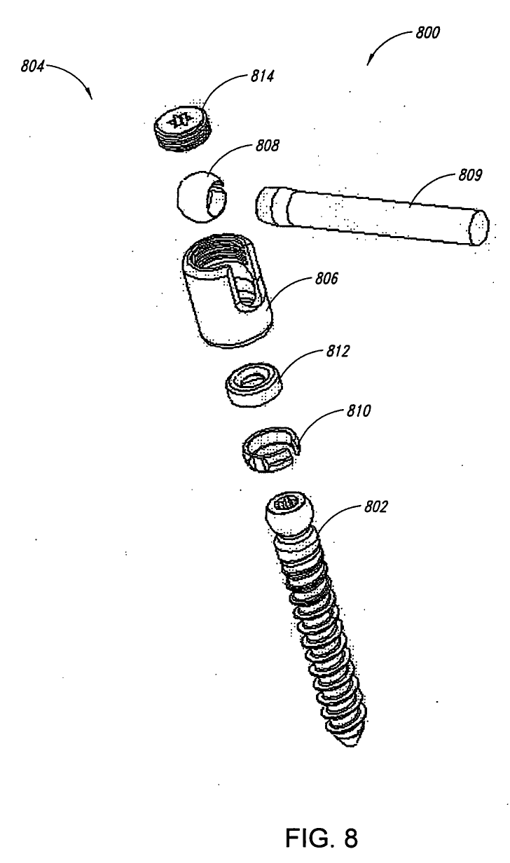

FIG. 8 illustrates yet another exemplary pedicle screw system (800) having a pedicle screw (802) and a tulip assembly (804), according to one exemplary embodiment. As illustrated in FIG. 8, the exemplary tulip assembly (804) includes a tulip body (806), a rod receiving member (808), a split ring (810), a saddle (812), and a set screw (814). The tulip assembly (804) of the present exemplary pedicle screw system can be provided, during surgery, with the rod receiving member (808) and a rod (809), pre-assembled with the tulip body (804). During surgery, the pedicle screw (802) receives the tulip body (804). In the illustrated embodiment, the tulip body (804) includes an opening (816) on only one side of the cylindrical wall (818) of the tulip body (804). Additional aspects of the exemplary tulip assembly as described herein and illustrated in FIG. 8 can be found in U.S. Patent Application No. 11/388,567, which application is incorporated by reference herein in its entirety.

The various embodiment of pedicle screws and/or pedicle screw systems, in particular the various tulip assemblies, have been provided and described for illustrative and exemplary purposes. The illustrated embodiments are not intended to limit or narrow the scope of the claims. It is understood and appreciated that other pedicle screw systems, pedicle screws, and/or tulip assemblies, not illustrated herein or some combination of those illustrated herein, can meet the spirit and scope of the claimed systems and methods.

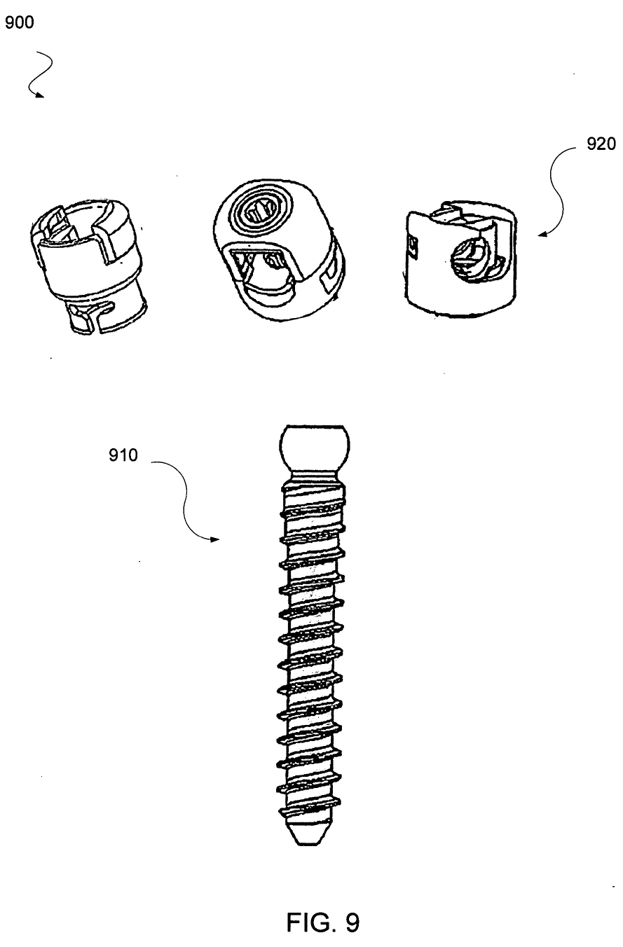

As mentioned previously, the present exemplary system and method includes a kit (900) illustrated in FIG. 9 having a pedicle screw (910) according to the exemplary embodiments illustrated above. Additionally, according to the present exemplary embodiment, a plurality of tulip assemblies (920) configured to be functionally coupled to the pedicle screw (910) is included in the kit (900). In contrast to traditional pedicle screw systems, the present exemplary system (900) is modular in that a plurality of the above-mentioned tulip assemblies (920) are configured to functionally couple a single pedicle screw (910), thereby providing a surgeon with the ability to intra-operatively determine which tulip assembly would be most beneficial to a particular patient's situation. Particularly, according to one exemplary embodiment, the present exemplary modular pedicle screw kit (900) allows the surgeon to leverage the various advantages of the above-mentioned tulip assemblies to benefit the patient.

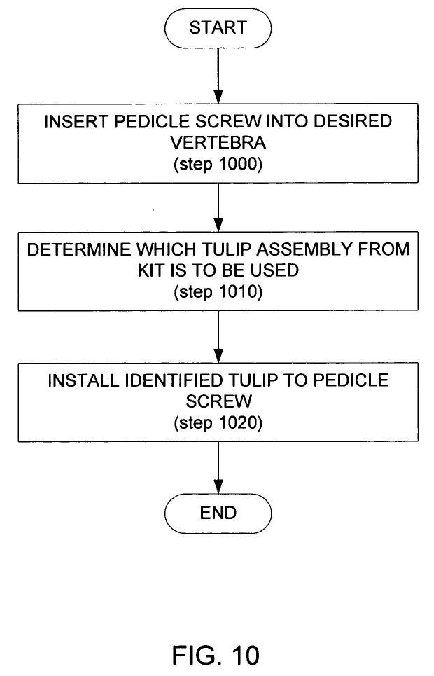

FIG. 10 illustrates an exemplary modular pedicle screw kit installation method, according to one exemplary embodiment. As illustrated in FIG. 10, the exemplary method may begin by inserting a pedicle screw into a vertebra (step 1000). According to one exemplary embodiment, the pedicle screw may be inserted percutaneously, through a minimally invasive tube, or through an incision.

Once the pedicle screw is inserted into a vertebra, the surgeon may determine a type of tulip assembly is to be compressively coupled to the head portion of the pedicle screw (step 1010). According to one exemplary embodiment, the surgeon may select any number of the above-mentioned exemplary tulip assemblies, or a number of other similar compressively coupling members. Consequently, the surgeon may select the tulip assembly that will best serve the patient's situation. Once selected, the identified tulip assembly may then be installed (step 1020).

Various embodiments of the present assemblies, devices, and systems have been described herein. It should be recognized, however, that these embodiments are merely illustrative of the principles of the present assemblies, devices, and systems. Numerous modifications and adaptations thereof will be apparent to those skilled in the art without departing from the spirit and scope of the present assemblies, devices, and systems.

The various embodiments described above can be combined to provide further embodiments. All of the above U.S. patents, patent applications and publications referred to in this specification as well as U.S. Provisional Patent Application No. 60/622,107 filed on Oct. 25, 2004, entitled CLIP LOCK PEDICLE SCREW; U.S. Provisional Patent Application No. 60/622,180 filed on Oct. 25, 2004, entitled POLY-AXIAL PEDICLE SCREW; U.S. Provisional Patent Application No. 60/629,785 filed on Nov. 19, 2004, entitled POLY-AXIAL PEDICLE SCREW; U.S. Provisional Patent Application No. 60/663,092 filed on Mar. 18, 2005, entitled MASTER LOCK PEDICLE SCREW; U.S. Provisional Patent Application No. 60/684,697 filed on May 25, 2005, entitled POLY AXIAL PEDICLE SCREW; and U.S. Provisional Patent Application No. 60/665,032, filed on Mar. 23, 2005, entitled PERCUTANEOUS PEDICLE SCREW SYSTEM, are incorporated herein by reference, in their entirety. Aspects of the exemplary system and method can be modified, if desired, to employ devices, features, and concepts of the various patents, applications and publications to provide yet further embodiments of the system and method.

In conclusion, the present exemplary systems and methods provide for modular pedicle screw systems and methods. Particularly, the present exemplary system and methods incorporate a number of pedicle screws and/or tulip assemblies configured to be combined to create pedicle screw kits that may be advantageously modified and assembled intra-operatively. Consequently, the present exemplary system and method provides a surgeon the option to vary their tulip selection once the surgical site is visually evaluated.

The preceding description has been presented only to illustrate and describe the present method and system. It is not intended to be exhaustive or to limit the present system and method to any precise form disclosed. Many modifications and variations are possible in light of the above teaching.

The foregoing embodiments were chosen and described in order to illustrate principles of the system and method as well as some practical applications. The preceding description enables others skilled in the art to utilize the method and system in various embodiments and with various modifications as are suited to the particular use contemplated. It is intended that the scope of the present exemplary system and method be defined by the following claims.

What we claim is:

1. A modular pedicle screw kit, comprising:
a pedicle screw having a threaded, elongated shaft coupled to a head portion; and
a plurality of tulip assemblies configured to be installed on said head portion of said pedicle screw;
wherein each of said plurality of tulip assemblies includes a tulip body, a rod receiving portion, and a coupling member configured to compressively couple said tulip body to said pedicle screw after said pedicle screw is inserted in a vertebra.

2. The modular pedicle screw kit of claim 1, wherein said rod receiving portion is positioned in said tulip body after installation of said tulip assembly onto said head portion of said pedicle screw.

3. The modular pedicle screw kit of claim 1, wherein said coupling member comprises a split ball configured to be positioned over said head portion of said pedicle screw.

4. The modular pedicle screw kit of claim 1, wherein said coupling member comprises a split ring configured to be positioned over said head portion of said pedicle screw.

5. The modular pedicle screw kit of claim 1, wherein said coupling member comprises a hoop device configured to be positioned over said tulip body to secure said tulip body to said head portion of said pedicle screw.

6. The modular pedicle screw kit of claim 1, wherein said head portion of said pedicle screw is generally spherical.

7. A method for intra-operatively assembling a pedicle screw kit, comprising:
inserting a pedicle screw into a vertebra, the pedicle screw having a threaded, elongated shaft coupled to a head portion;
selecting one type of tulip assembly from a plurality of tulip assemblies to be installed on the head portion of the pedicle screw after the pedicle screw is inserted in the vertebra, wherein each of said plurality of tulip assemblies includes a tulip body, a rod receiving portion, and a coupling member configured to compressively couple said tulip body to said pedicle screw; and
coupling said selected one type of tulip assembly to said head portion of said pedicle screw.

8. The method of claim 7, wherein selecting the one type of tulip assembly to be coupled to the head portion of the pedicle screw comprises verifying said one type of tulip assembly originally selected as determined from a non-invasive imaging procedure.

9. The method of claim 7, further comprising examining said vertebra via a non-invasive imaging procedure before opening a surgical site.

10. The method of claim 7, wherein coupling said one type of tulip assembly to said head portion of said pedicle screw comprises affixing a portion of said tulip body on said head portion of said pedicle screw.

11. The method of claim 7, wherein determining said one type of tulip assembly includes selecting said coupling member to be a split ball.

12. The method of claim 11, wherein coupling said one type of tulip assembly to said head portion of said pedicle screw comprises:
positioning said split ball over said head portion of said pedicle screw; and
compressively engaging said tulip body with said split ball to releasably couple said tulip body to the pedicle screw.

13. The method of claim 7, wherein determining said one type of tulip assembly comprises selecting said coupling member to be a split ring.

14. The method of claim 13, wherein coupling said one type of tulip assembly to said head portion of said pedicle screw comprises:
positioning said split ring over said head portion of said pedicle screw; and
engaging said tulip body with said split ring to releasably couple said tulip body to said pedicle screw.

15. The method of claim 7, wherein determining said one type of tulip assembly comprises selecting the coupling member to be a hoop.

16. The method of claim 15, wherein coupling said one type of tulip assembly to said head portion of said pedicle screw comprises:
positioning said hoop over said tulip body after said tulip body has received said head portion of said pedicle screw; and
rotating said hoop to releasably couple said tulip body to said pedicle screw.

17. The method of claim 7, wherein coupling said type of tulip assembly to said head portion of said pedicle screw comprises inserting a rod into said rod receiving portion.

18. A modular pedicle screw kit, comprising:
a pedicle screw having a threaded, elongated shaft coupled to a head portion; and
a plurality of tulip assemblies configured to be compressively coupled to said head portion of said pedicle screw.

19. The modular pedicle screw kit of claim 18, wherein each of said plurality of tulip assemblies includes a tulip body, a rod receiving portion, and a coupling member configured to compressively couple said tulip body to said pedicle screw.

20. The modular pedicle screw kit of claim 18, wherein each of said plurality of tulip assemblies is configured to be compressively coupled to said head portion of said pedicle screw after said pedicle screw is inserted in a vertebra.
